(19) United States
(12) Patent Application Publication
(10) Pub. No.: US 2025/0360283 A1
(43) Pub. Date: Nov. 27, 2025

(54) AROMATHERAPY DEVICE

(71) Applicant: LIFELINES, LLC, Westport, CT (US)

(72) Inventors: Tyler Smith, Baltimore, MD (US); John Vincent Abram, Oceanside, CA (US); Justin Riley, Needham, MA (US); Melissa Bernstein, Westport, CT (US)

(21) Appl. No.: 18/673,777

(22) Filed: May 24, 2024

(51) Int. Cl. A61M 21/00; B43K 24/02; B43K 29/20

(52) U.S. Cl. CPC A61M 21/00; B43K 24/02; B43K 29/20; A61M 2021/0016

(57) ABSTRACT

An aromatherapy device is formed by a cartridge body connected to a writing instrument, such as a pen. The cartridge body forms a plurality of reservoirs, each adapted to hold a volatile, scented liquid. Distal ends of a respective plurality of wicks extend into each of the reservoirs. Proximal ends of the wicks extend into a vent housing. The vent housing has a plurality of vent openings, each positioned adjacent the proximal end of one of the wicks. Each vent opening has a vent actuator that allows a user to selectively open and close the vent. Liquid is transported by the wick from the reservoir into the vent housing. When the user opens one or more of the vents, liquid evaporates from the wick and the vapor escapes from the device to provide a source for an aromatherapy treatment, for example, while the user is writing with the writing instrument.

### BACKGROUND OF THE INVENTION

### 1. Field of the Invention

The present disclosure relates to a scent emitter integrated with a writing instrument, for example a pen or mechanical pencil. More particularly, the present disclosure relates to an aroma emitting assembly connected with a pen where one or more scent emitting chambers can be selectively opened to allow scent generated by a volatile liquid to diffuse from the apparatus.

### 2. Description of the Related Art

Aromatherapy is a technique for improving emotional and/or physical health of a person by providing aromatic scents. Such scents may be provided by evaporating volatile liquids such as essential oils. Having a source of such scents readily available may improve a user's mood and health. Providing a source of scents near to where a person is writing may be particularly useful.

Aroma may have influence on a person's implicit memory and mental performance as discussed in Odors: Implicit Memory and Performance Effects, Chemical Senses, Oxford University Press, UK. According to the article, 108 subjects were randomly divided into three groups and entered three different spaces in which a jasmine essential oil, a lavender essential oil, and no essential oil were respectively diffused. The group corresponding to the use of no essential oil was the control group. The experiment lasted for 30 minutes, during which the subjects were required to complete arithmetic and spelling tests. The results of the experiment show that the answer rate and correct answer rate of the two essential oil groups were higher than those of the control group. While not wishing to be bound by theory, it has been suggested that providing selected scents to a user may allow the user to better concentrate on difficult tasks.

Scents may also help a person relax during a stressful time, such as while writing documents. Scents may help inspire a person while writing for enjoyment. Scents may evoke a sense of well-being while writing, for example, in a personal journal or while drawing or painting. Thus, there is a need for a scent emitter that is conveniently accessible to a person using a writing instrument such as a pen, pencil, or paint brush.

Aromatherapy techniques may rely on providing a user with different scents at different times of the day, while the user is performing different kinds of tasks, or when the user's mood is best treated by providing a particular scent or combination of scents. For example, when a user feels tired, a scent such as menthol may help awaken the user to continue a difficult or tiresome task. Or where a user feels stressed, a scent such as lavender may help the user relax. Thus, there is a need for a device that provides selectable sources of a number of different scents.

Scents generated by evaporating volatile fluids such as essential oils may dissipate quickly. The farther away from the source of the evaporating fluid, the less intense the scent may be. Thus, there is a need for a scent emitter that can be conveniently located close to the user.

Volatile fluids used to generate aromatherapy scents, such as essential oils, may be expensive. Such oils may be prone to stain clothing and surfaces if they are not properly contained. Thus, there is a need to provide a scent emitter that reliably and securely holds volatile fluids and only allows vapors to escape to prevent wasting such valuable fluids and damaging a user's belongings.

Different users may have different sensitivities to different scents. Also, different volatile fluids used to generate aromatherapy scents have different vapor pressures, making the scents generated by one fluid more or less intense. A user may wish to vary the intensity of a particular scent. Thus, there is a need for a scent generator that allows a user to select how rapidly an aroma generating fluid evaporates.

### BRIEF SUMMARY OF THE INVENTION

Embodiments of the disclosure address these and other needs. According to one embodiment of the disclosure, there is provided an aroma emitter connected with a writing instrument, such as a pen, mechanical pencil, paintbrush and the like. Such an aroma emitter includes a reservoir for holding a volatile substance that evaporates to provide a scent. According to one embodiment, a wick extends from the reservoir to a cartridge housing. Capillary action moves the substance along the wick and into a cartridge housing. The cartridge housing includes a vent opening and a vent actuator. The vent actuator may be a slider that allows the user to selectively open and close the vent opening to allow vapors generated by the evaporating volatile substance to escape to be sensed by the user, for example, as an aromatherapy treatment.

According to one embodiment the device includes a plurality of reservoirs holding a respective plurality of different volatile substances to provide a user with different scents or combinations of scents. Separate wicks are inserted into respective ones of the reservoirs to deliver the substance to separate compartments of the cartridge housing. Each compartment has a separate vent opening and vent actuator. A user can select one or more vent actuators to allow different scents or combinations of scents to be emitted.

### BRIEF DESCRIPTION OF THE DRAWINGS

The above and/or other aspects of the disclosure will be more apparent by describing in detail exemplary embodiments of the disclosure with reference to the accompanying drawings, in which:

FIGS. 1 is a perspective view of a writing instrument and scent emitter according to an embodiment of the disclosure;

FIG. 2 is an exploded view of the writing instrument and scent emitter of FIG. 1;

FIG. 3 is an exploded view of the writing instrument portion of the device of FIG. 1;

FIG. 4 is an exploded view of the scent emitter of FIG. 1;

FIG. 5 is an exploded view of a cartridge cap assembly forming a component of the scent emitter of FIG. 4;

FIGS. 6 is a detailed perspective view of a wick forming a component of the scent emitter of FIG. 4;

FIG. 7 is a perspective view of a cartridge vent housing forming a component of the scent emitter of FIG. 4;

FIG. 8 is a perspective view of a cartridge cap forming a component of the scent emitter of FIG. 4;

FIG. 9 is a perspective view of a vent actuator forming a component of the scent emitter of FIG. 4;

FIG. 10 are perspective views of embodiments of the vent actuator of FIG. 9;

FIGS. 11A and 11B are a perspective view and a bottom view, respectively, of a cartridge vent housing according to a further embodiment of the disclosure;

FIG. 12A is a perspective view of a scent emitter according to a still further embodiment of the disclosure; and

FIG. 12B is a vent plug forming a component of the scent emitter of FIG. 12A.

### DETAILED DESCRIPTION OF EXEMPLARY EMBODIMENTS

Exemplary embodiments of the disclosure will now be described below by reference to the attached Figures. The described exemplary embodiments are intended to assist the understanding of the invention and are not intended to limit the scope of the invention in any way. Like reference numerals refer to like elements throughout.

The term “distal” refers to the direction away from a user operating apparatus according to the disclosure. The term “proximal” refers to the direction toward a user operating apparatus according to the disclosure.

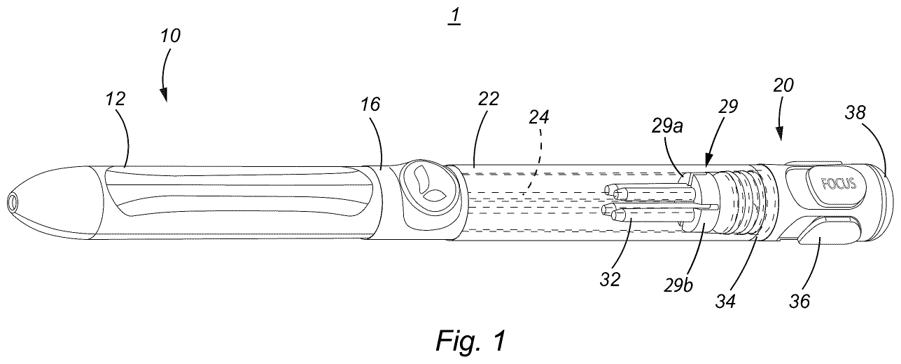

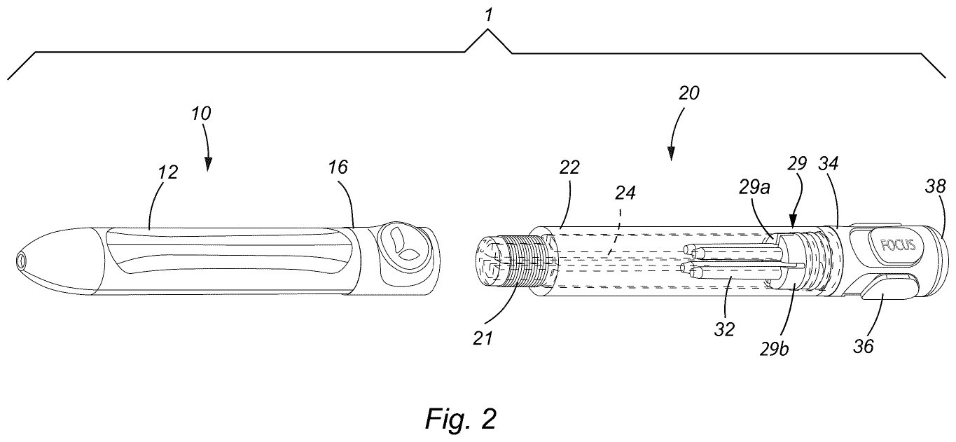

FIGS. 1 and 2 show a scent emitter and writing instrument 1 according to one embodiment of the disclosure. A scent emitter assembly 20 is connected with a writing instrument assembly 10. As shown in FIG. 2, the scent emitter assembly 20 may include a connection 21 adapted to connect the scent emitter 20 with the writing instrument assembly 10. According to one embodiment, connection 21 has a screw thread shaped to rotatably engage with a corresponding threaded portion of writing instrument assembly 10. In another embodiment, connection 21 is formed by a friction fit, a snap fit interface, by a layer of adhesive, by a weld, by a fastener, or by other connection mechanisms known to those of skill in the field of the disclosure. According to one embodiment, connection 21 releasably joins assemblies 10 and 20. According to another embodiment, connection 21 allows assemblies 10 and 20 to be disconnected, for example, where the writing instrument assembly is to be replaced, such as when a supply of ink is exhausted. According to another embodiment, scent emitter 20 and instrument assembly 10 are permanently fixed together.

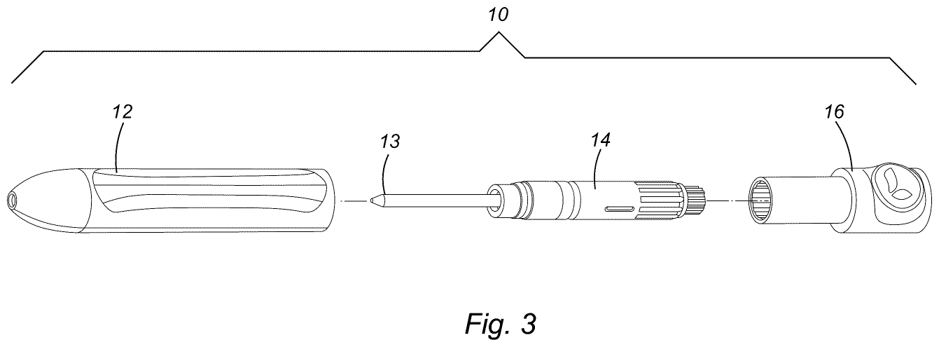

Referring now to FIG. 3, an exemplary embodiment of the writing instrument assembly 10 according to the present disclosure is shown. Writing instrument assembly 10 includes a pen body 12, a marking device 13, an actuating mechanism 14, and a knob 16. Actuating mechanism 14 and marking device 13 are inserted into the pen body 12 so that a marking device 13, for example, a ball point pen cartridge, extends coaxially thought the pen body and extends through an opening at the distal end of the pen body. The proximal end of the pen body 12 is configured to accept the distal end of the knob 16. In a preferred embodiment, the actuating mechanism 14 includes a mechanism for moving the marking device 13 proximally and distally with respect to pen body 12 so that the marking device can be selectively extended to protrude from the distal end of the pen body 12 and retracted so that the distal end of the marking device 13 is housed within the pen body 12. According to one embodiment, the actuating mechanism 14 includes a rotatable mechanism that engages with the knob 16 so that rotation of the knob relative to the pen body 12 in a first direction extends the marking device 13 from the pen body and rotation in a second direction retracts the marking device 13 into the pen body 12. According to one embodiment, the rotatable mechanism includes a helical cam and cam follower (not shown) that translate rotational motion of the knob 16 into linear motion of marking device 13. In other embodiments, the marking device 13 may be extended and retracted by other means known to those of skill in the field of the invention. According to other embodiments, marking device 13 is in a fixed position with respect to the pen body 12 so that the marking device 13 constantly protrudes through the distal end of the pen body 12. The marking device 13 may be an ink pen, a ball point pen, a gel pen, a felt tip pen, a pencil, a crayon, a brush, such as a paint brush, a metal alloy stylus or calligraphy brush or the like. According to one embodiment, marking device 13 comprises a metal tip or stylus formed from an alloy that creates marks on paper or other surfaces without appreciable wear to the tip. Such a tip, sometimes called an “infinite pencil,” an “eternal pencil” or an “ink-less pen” may be fixed at the distal end of pen body 12 or else connected with actuating mechanism 14 to be extended and retracted from the pen body.

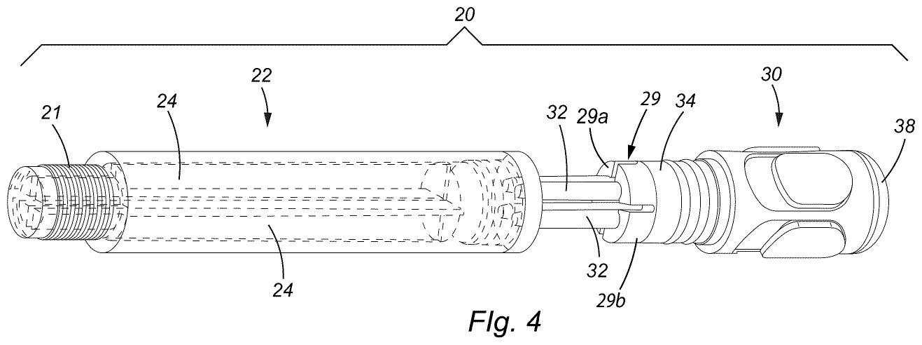

Referring to FIG. 4, an exploded diagram of an exemplary embodiment of the scent emitter assembly 20 is shown. The scent emitter assembly 20 includes a cartridge body 22 with at least one reservoir 24 and a cartridge cap assembly 30. A seal 34 at the distal end of the cartridge cap assembly 20 connects the cap assembly with the proximal end of the cartridge body 22. The cartridge body 22 may be permanently or preferably non-permanently coupled with the distal end of the cartridge cap assembly 30. According to some embodiments, cartridge cap assembly 30 is connected with cartridge body 22 by adhesives, welds, friction fit, or by a fastener so that a sealed connection joint exists between the cartridge body 22 and the cartridge cap assembly 30.

The cartridge body 22 has at least one reservoir 24 configured to store a volatile scented liquid. In the embodiment of FIG. 4, four reservoirs 24 are provided to each hold a separate liquid. According to one embodiment, reservoirs 24 are formed in the cartridge body 22 with their proximal ends open before the cap assembly 30 is sealed via seal 34 with the cartridge body. Selected liquids are put in the reservoirs 24 through the respective open ends of the reservoirs 24 when the cap assembly 30 is separated from the cartridge body 22. When the cap assembly 30 is connected with the cartridge body 22, the distal face of the seal 34 presses against the open ends of the reservoirs 24 to keep liquids in each of the reservoirs separate from one another.

According to one embodiment, gasket 29 is provided between cartridge body 22 and seal 34 for creating a seal between the reservoir 24 and seal 34 to ensure volatile liquids remain in their respective reservoir 24. In one exemplary embodiment, the gasket 29 is a separate component having a distal end 29a that is at least partially inserted into the reservoir 24, a proximal end 29b that abuts the seal 34 and at least one through hole 29c extending through the distal end of the gasket 29. Distal end 29a of gasket 29 is dimensioned such that a liquid tight fit is achieved between the reservoir 24 and the gasket 29. In the illustrated embodiment shown in FIGS. 1, 2 and 4, there are four through holes 29c that allow for a wick 32 to be inserted into a respective reservoir 24. However, a greater or fewer number of through holes 29c may be used. A close fit between a distal extension 32a of a wick 32 and the respective through hole 29c prevents liquid from flowing out of the reservoir 24 around the wick and thus limits the flow of the liquid only to capillary flow through the wick. It is also contemplated within this disclosure that gasket 29 can be integrally formed as one piece of the distal end of the cap assembly 30.

According to one embodiment, seal 34 removably connects the cap assembly 30 with the cartridge body 22. A user can refill one or more of the reservoirs 24 by removing the cap assembly 30 and gasket 29 from the cartridge body 22 and filling the reservoirs through the open ends of the reservoirs. In one exemplary embodiment four reservoirs are provided. However, a greater or fewer number of reservoirs 24 may be provided within the scope of this disclosure.

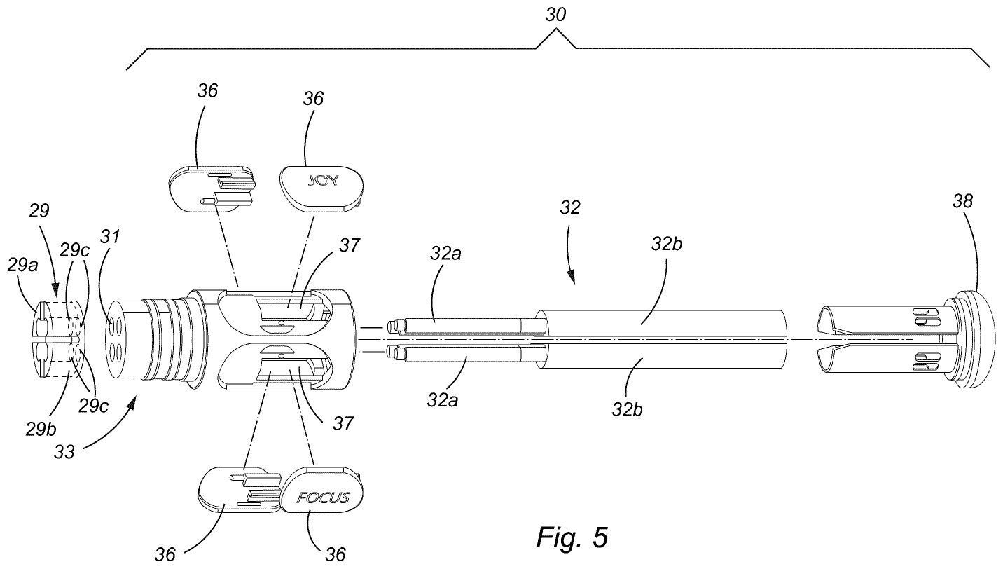

Referring to FIGS. 4 and 5, an exemplary embodiment of the cartridge cap assembly 30 is shown. The cartridge cap assembly 30 includes at least one wick 32, a cartridge vent housing 33, at least one vent 37, at least one vent actuator 36, and a cartridge cap 38. In the embodiment of FIGS. 4 and 5, four such wicks 32 are shown extending into four respective reservoirs 24, but a greater or fewer numbers of wicks and reservoirs may be provided with the scope of the disclosure. In this exemplary embodiment, the cartridge vent housing 33 includes through holes 31 extending through the distal end of the cartridge vent housing.

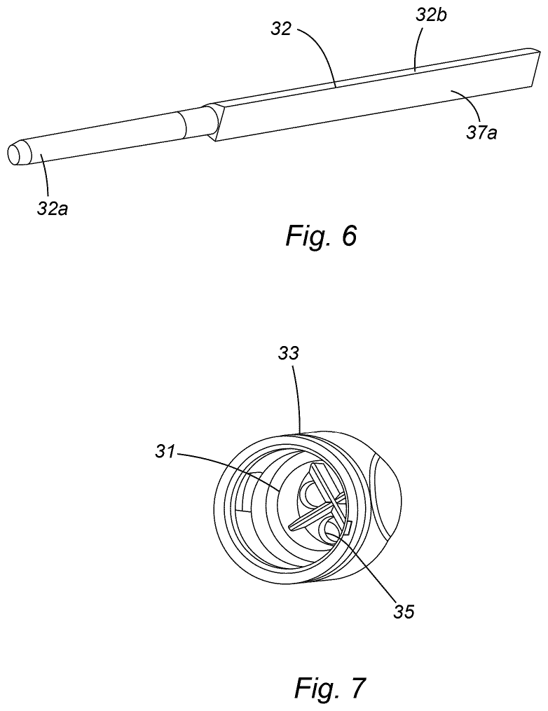

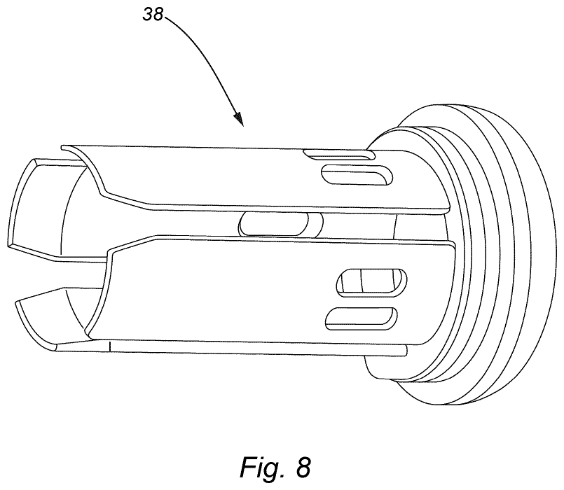

FIG. 6 shows a detailed view of one of the wicks 32. FIG. 7 shows a detailed perspective view of vent housing 33. Each wick 32 includes a distal extension 32a sized to fit closely within through holes 31 in the cartridge vent housing 33 and extend into a respective one of the reservoirs 24. A close fit between the distal extensions 32a and the through holes 31 prevents liquid from flowing out of the reservoir 24 around the wick and thus limits flow of the liquid only to capillary flow through the wick. In the exemplary embodiment, four reservoirs 24 are provided with four wicks 32 that each correspond with one of the four reservoirs 24. A greater or fewer number of wicks 32 may be used within the scope of this disclosure.

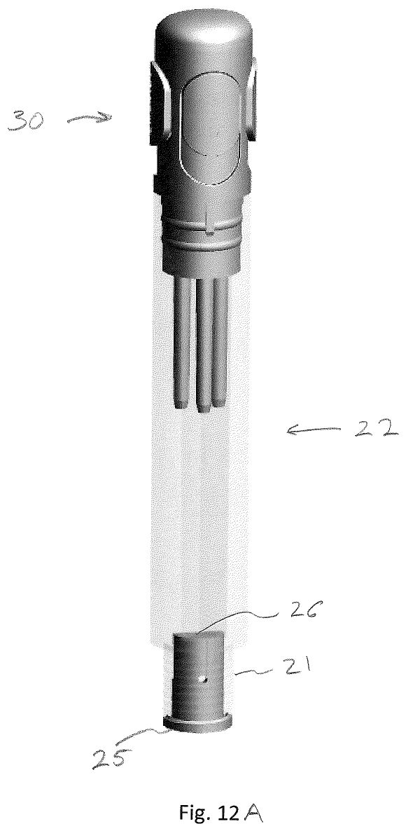

FIGS. 11A and 11B show vent housing 33 according to an alternate embodiment of the disclosure. As discussed above, wicks 32 are shaped to form a close fit with holes 31 so that the flow of liquid from the reservoir 24 into vent housing 33 takes place only within the wick and not along the outer surface of the wick. In some embodiments, the flow of liquid out from reservoir 24 by capillary action through wick 32 may create a partial vacuum within the reservoir. This partial vacuum may restrict the flow of liquid through the wick. According to the embodiment of FIGS. 11A and 11B, holes 31 include one or more extensions 31a that extend radially outward from the hole. Extensions 31a form gaps between the edge of hole 31 and the surface of distal extension 32a of the wick to provide a passage for air to flow into the respective reservoir 24 to equilibrate the reservoir 24 with the ambient atmospheric pressure. According to one embodiment, the size of extensions 31a, and hence the gap is selected so that the viscosity of the liquid in reservoir 24 prevents the liquid from flowing through the gap. According to another embodiment, in addition to the gap size, the material used to form vent housing 33, or at least the portion of vent housing 33 adjacent to holes 31 has surface characteristics (e.g. hydrophobicity, lipophobicity) such that the material is not wetted by the liquid or is wetted only to a limited extent so that surface tension prevents the liquid from flowing through the gaps provided by extensions 31a.

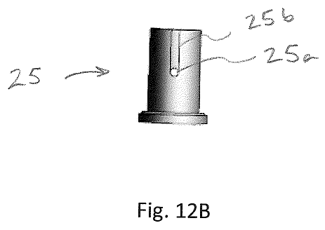

FIGS. 12A and 12B show a further alternate embodiment of the disclosure. In this embodiment cartridge body 22 is formed so that reservoirs 24 are open at both proximal and distal ends. The distal ends of reservoirs 24 are closed by seal 34 on cartridge vent housing 33, as described in the embodiments discussed above. Openings at the proximal ends of reservoirs 24 are closed by vent plug 25. Vent plug 25 is inserted into the open distal end of cartridge body 22. Vent plug 25 includes a proximal surface 26 that is shaped to contact distal ends of the reservoirs 24 and form a liquid-tight seal across the distal ends of the reservoirs 24. Plug 25 includes holes 25a that extend through the sides of the plug and communicate with a central space within the writing instrument assembly 10. Writing instrument assembly 10 is not sealed, so that holes 25a communicate with the ambient atmosphere. Slots 25b extend along the outer surface of plug 25 and through proximal surface 26 so that the proximal ends of slots 25b are in communication with respective ones of the reservoirs 24. Dimensions of holes 25a and slots 25b, as well as wetting characteristics of the material forming plug 25, may be chosen to prevent liquid in the reservoir from flowing out through the slots and holes, while providing a path for air to flow into the reservoir to equilibrate the pressure inside the reservoir 24 with the ambient atmosphere.

Turning back to FIGS. 4 and 5, the cartridge vent housing 33 has a plurality of vents 37. In one embodiment, each of the vents 37 corresponds with a respective one of the reservoirs 24 and wicks 32. In another embodiment, a plurality of vents 37 may correspond to a plurality of wicks 32 extending into a respective one of the reservoirs 24. The cartridge vent housing 33 has four vents 37. A greater or fewer number of vents 37 may be used within the scope of this disclosure.

According to one embodiment, interior walls 31 are provided in a vent housing 33 as shown in FIG. 7. Walls 31 separate proximal portions 32b of the wicks 24. Walls 31 prevent liquids or vapors from each wick 24 from mixing with one another. According to another embodiment, as shown in FIG. 6, an impenetrable layer 37a is provided on proximal portions 32b of the wicks 32. When wicks 32 are assembled, as shown for example in FIG. 5, the impenetrable layers 37a prevent liquid or vapor from adjacent wicks 32 from mixing inside the vent assembly.

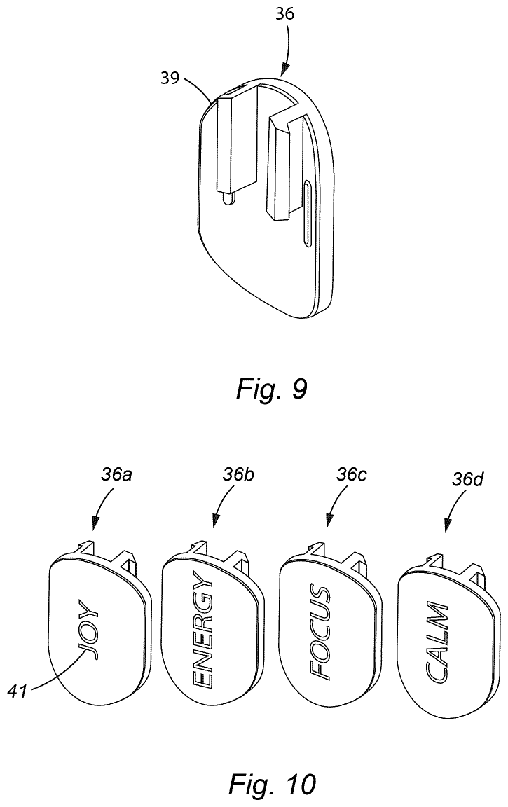

Proximal ends 32b of the wicks 32 are positioned within the vent housing 33 adjacent a respective one of the vents 37. A cartridge cap 38 is couple to the proximal end of the cartridge vent housing 33 to secure the wicks 32 in the cartridge vent housing 33 and to form closed spaces around proximal ends 32b of each wick 32. FIG. 8 shows a detailed view of cap 38.

According to one embodiment, each of the vents 37 is provided with a vent actuator 36 to selectively open and close the vent. In the embodiment of FIG. 5, the vent actuators 36 are sliders that can be moved proximally and distally along the vent housing 33. When an actuator is moved in a first, distal direction, the respective vent 37 is opened to the outside air, exposing the proximal end 32b of the respective wick 32. Volatile liquid carried from the respective one of the reservoirs 24 by capillary action of the wick 32 evaporates from the proximal end 32b of the wick and exits the vent to be enjoyed by the user. When the slider 36 is moved in a second, proximal direction, the respective vent 37 is closed, preventing or at least significantly reducing the transmission of vapor from the respective wick 32 through the vent. It is to be appreciated that other types of actuators may be used to open and close the vents 37. For example, vent actuators 36 could be connected with the cartridge assembly 30 by hinges to swing away from the vents 37 to open them. According to another embodiment, vents 37 are opened and closed by a cylinder having one or more openings that is rotateably disposed on the outside surface of the assembly so that alignment of the holes in the cylinder with one or more of the vents 37 opens and closes vents 37.

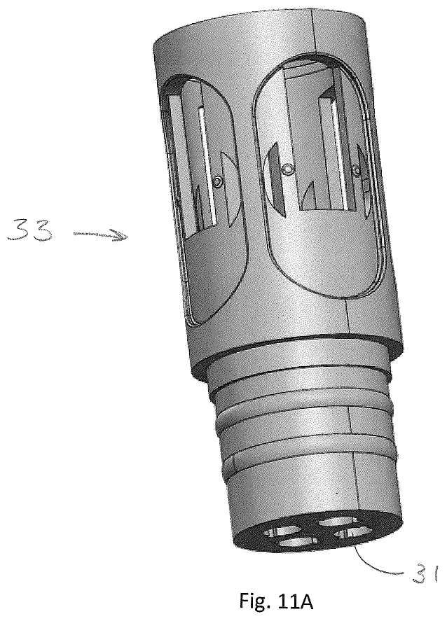

FIG. 9 shows a detailed view of a slider 36. According to one embodiment, Slider 36 includes one or more tabs 39 extending along the inside surface of the slider. Tab 39 engages with the sides of vent 37 to allow the slider to move proximally and distally along vent housing 33. According to one embodiment, detents are provided on the slider 36 and/or vent housing 33 to enable the sliders to be releasably held in an open or closed configuration. According to a further embodiment, a plurality of detents are provided to allow the slider 36 to be positioned at intermediate positions between full opened and fully closed to allow a user to modulate the amount of aromatic vapors emitted via the vent.

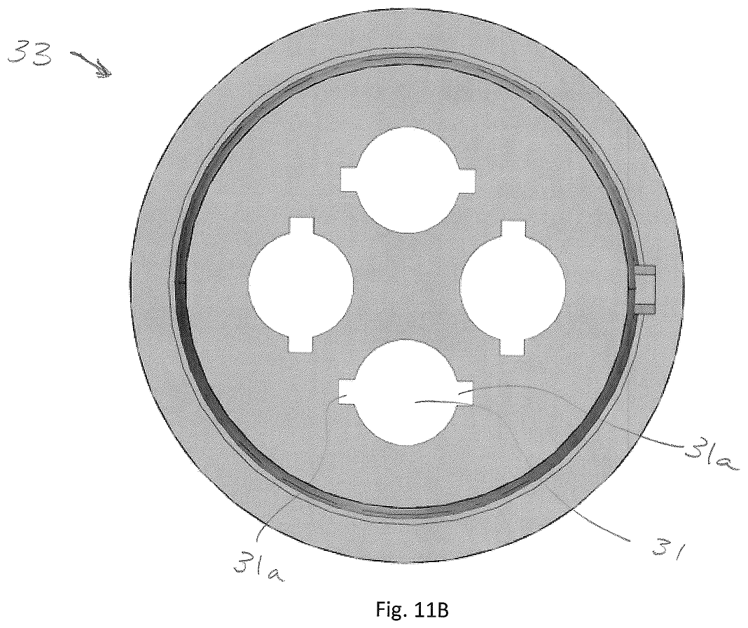

Sliders 36 may be provided with indicia 41 on their outside surface to inform the user which vent 37 corresponds to the reservoir 24 holding a selected scented liquid. In the embodiment of FIG. 10, indicia 41 include words or symbols corresponding to particular aromas provided from each of the reservoirs for each slider 36a-36d. According to other embodiments, instead of or in addition to word or symbols, indicia 41 include tactile features to allow a user to select an aroma by touch.

Wick 32 is made from a material that is suitable to allow the fluid to be transported through the wick 32 by capillary action. Suitable materials include natural or synthetic fibers, wood, bone, cloth, and sintered materials such as sintered ceramics, sintered metals, and sintered polymers. In a preferred embodiment, the wick 32 is made of sintered polypropylene.

While the invention has been particularly shown and described with reference to exemplary embodiments thereof, the invention is not limited to these embodiments. It will be understood by those of ordinary skill in the art that various changes in form and details may be made therein without departing from the spirit and scope of the invention as defined by the following claims. Therefore, the description should not be construed as limiting the scope of the invention.

What is claimed is:

1. An aromatherapy device comprising:
a writing instrument;
a cartridge body connected with the writing instrument and defining at least one reservoir adapted to contain a volatile substance;
a cartridge cap connected with the cartridge body and defining at least one vent opening;
at least one wick, wherein a proximal portion of the wick is disposed within the cartridge cap adjacent the opening and wherein a distal portion of the wick is disposed in the reservoir; and
at least one vent actuator movably connected with the cartridge cap adjacent a respective one of the at least one vent, wherein, in a first configuration the vent actuator opens the respective vent opening allowing the volatile liquid to evaporate and release vapors through the vent opening and in a second configuration the vent actuator closes the respective vent opening.

2. The aromatherapy device of claim 1, wherein the at least one vent actuator comprises a slider disposed adjacent the vent opening and slidable in a proximal and a distal direction.

3. The aromatherapy device of claim 1, wherein the at least one vent opening comprises a plurality of vent openings, wherein the at least one reservoir comprises a respective plurality of reservoirs, wherein the at least one wick comprises a respective plurality of wicks, wherein the at least one vent opening comprises a respective plurality of vent openings, and wherein the at least one vent actuator comprises a respective plurality of vent actuators.

4. The aromatherapy device of claim 3, wherein the plurality of vent actuators each comprise an indicia.

5. The aromatherapy device of claim 4, wherein the indicia comprise one or more of words, symbols and tactile features.

6. The aromatherapy device of claim 1, wherein the volatile substance is an essential oil.

7. The aromatherapy device of claim 3, wherein each of the plurality of reservoirs holds a different volatile substance.

8. The aromatherapy device of claim 1, wherein the writing instrument comprises a tube coupled with the proximal end of the cartridge body and a marking device disposed within the tube.

9. The aromatherapy device of claim 8, wherein a distal end of the marking device extends from a distal opening at a distal end of the tube.

10. The aromatherapy device of claim 8, further comprising an actuation mechanism connected with the tube and the marking device, wherein actuation of the actuation mechanism moves the marking device from an extended configuration wherein the marking device extends from a distal end of the tube and a retracted configuration wherein the marking device is retracted within the tube.

11. The aromatherapy device of claim 8, wherein the writing instrument comprises an ink pen, a ball point pen, a gel pen, a felt tip pen, a pencil, a crayon, an inkless pen, a paint brush, a metal alloy stylus, or a calligraphy brush.

12. The aromatherapy device of claim 1, wherein the cartridge cap comprises a wick hole, wherein the at least one wick is sized to form a liquid tight fit with the wick hole, wherein the volatile liquid is prevented from flowing into the cartridge cap between the wick and the wick hole.

13. The aromatherapy device of claim 12, wherein the wick hole further comprises one or more radial extensions, wherein the extensions create a gap between the cartridge cap and the wick, and wherein the gap is sized to allow air to flow through the gap and to prevent the volatile liquid from flowing through the gap.

14. The aromatherapy device of claim 1, wherein the cartridge body defines an distal opening at a distal end thereof, wherein the distal opening is in fluid communication with a distal end of the at least one reservoir, the device further comprising a plug sized to sealingly fit into the distal opening, the plug defining an air passage from an ambient atmosphere into the at least one reservoir, wherein at least a portion of the air passage is sized to prevent flow of the volatile liquid.
